(19) United States
(12) Patent Application Publication
(10) Pub. No.: US 2026/0049058 A1
(43) Pub. Date: Feb. 19, 2026

(54) CRYSTALLINE FORMS OF PICOLINAMIDE FUNGICIDE COMPOUND

(71) Applicant: CORTEVA AGRISCIENCE LLC, INDIANAPOLIS, IN (US)

(72) Inventors: CAROLINE LONG, INDIANAPOLIS, IN (US); ADRIANE MILLER, SAGINAW, MI (US); MARK P. MUEHLFELD, SOUTH HAVEN, MI (US); XIAOWEN ZHAO, SOMERVILLE, NJ (US); NICOLA WEBB, HOPEWELL, NJ (US)

(73) Assignee: CORTEVA AGRISCIENCE LLC, INDIANAPOLIS, IN (US)

(21) Appl. No.: 18/865,738

(22) PCT Filed: May 18, 2023
(86) PCT No.: PCT/US23/22639

§ 371 Date: Nov. 14, 2024

Related U.S. Application Data
(60) Provisional application No. 63/365,532, filed on May 31, 2022.

(51) Int. Cl. C07D 213/81; B01D 9/00

(52) U.S. Cl. CPC C07D 213/81; B01D 9/0018; B01D 9/0036

(57) ABSTRACT

The present technology relates to processes useful for making crystalline S)-1,1-bis(4-fluorophenyl)propan-2-yl (3-acetoxy-4-methoxypicolinoyl)-L-alaninate (Compound I). Processes disclosed herein describe more stable, storage compatible preparations of Compound I. Also disclosed are preparations of Compound I which have fewer (trace) impurities.

### FIELD

The present disclosure is related to the fields of crystalline forms of, compositions thereof, as well as uses thereof, and processes to make or manufacture crystalline forms of (S)-1,1-bis(4-fluorophenyl)propan-2-yl (3-acetoxy-4-methoxypicolinoyl)-L-alaninate (Compound I); which is a known compound that controls fungal diseases.

### BACKGROUND

Fungicides are compounds, of natural or synthetic origin, which act to protect and cure plants against damage caused by agriculturally-relevant fungi. Consequently, research is ongoing to find new fungicides or new forms thereof; and for processes thereof—which are desirable from the standpoint of increased biological activity, atom economy of manufacture, compositional chemical stability or other such benefits the improved fungicide (form/process) may confer.

The solid state of chemical compounds—in the present case, fungicidal compounds—can be amorphous (i.e. no long-range order in the positions of atoms) or crystalline (i.e. atoms arranged in an orderly repeating pattern). Research is ongoing to discover improved forms of chemical compounds such that compositions and/or neat preparations of the compound of interest can be stored, shipped and/or found to be more biologically active or possess more desirable physical characteristics such as a lower melting point, hydroscopicity, etc., or contain higher purity, i.e. less trace compounds detected (unwanted) in the preparation and thus, fewer impurities.

The present disclosure relates to a fungicide: crystalline forms of (S)-1,1-bis(4-fluorophenyl)propan-2-yl(3-acetoxy-4-methoxypicolinoyl)-L-alaninate (Compound I) and processes to make or manufacture crystalline forms thereof. Such forms are more stable, provide a cleaner impurity profile, and are active against and/or offer protection against ascomycetes, basidiomycetes, and deuteromycetes.

### SUMMARY

One aspect of the present disclosure provides for one or more crystalline forms of Compound I.

Compound I is unusually difficult to make in a crystalline form. The amorphous material isolated from working up an organic layer containing Compound I, either by column chromatography or by isolation of a salt, results in the formation of an agglomerated solid.

In fact, it was found that one tried and true method to increase the crystalline formation of an organic compound was not successful for Compound I. Specifically, increasing the oil concentration of a crude suspension containing Compound I (and seed) did not induce crystallization. The procedure followed was: an acid wash procedure was used on the crude reaction mixture containing Compound I, which created more oil during the crystallization. The temperature was then increased during a solvent exchange which increased the oil concentration as well. After the solvent exchange, the temperature was held at 30° C. for 2 hours, then cooled to 8° C. over 12 hours. The following morning, the reaction mixture had completely oiled. The resulting sample showed that the concentration was 7.02 wt % of Compound I. The mixture was readily redissolved, seeded with 1% seed Compound I (relative to Compound I in the mixture) at 25° C. The general temperature profile (holding at 25° C. for two hours, cooling to 8° C. over 12 hours) produced Compound I agglomerates stuck inside of the reactor. After initiating the filtration of the solids, the cake was washed and attempted to be collected. Unfortunately more agglomerates were observed throughout the filtered reaction slurry.

A stable form screen of Compound I was carried out. Many screening experiments produced either an oil or gel of Compound I. Two polymorphs were identified, designated as forms A and B. It was determined that the forms are monotropic. The melting point of each of the two crystalline forms is about 90° C., while the amorphous form exhibited a glass transition (Tg) event near 55° C. The crystalline materials are flowable.

Accordingly, one aspect of the present disclosure includes a process or method of manufacture to make one or more crystalline forms of Compound I. Further, one aspect of the present disclosure includes a process to make a composition of Compound I which has fewer (trace) impurities, i.e. a Compound I with high purity. One aspect of the present disclosure includes substantially pure Compound I.

The chemical structure of (S)-1,1-bis(4-fluorophenyl)propan-2-yl (3-acetoxy-4-methoxypicolinoyl)-L-alaninate (Compound I) is:

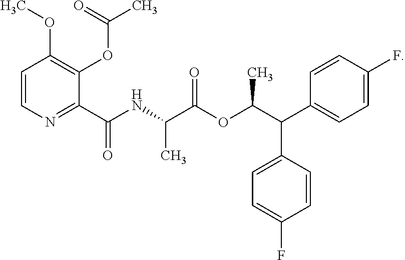 [SMILES: COc1ccnc(C(=O)N[C@@H](C)C(=O)O[C@@H](C)C(c2ccc(F)cc2)c2ccc(F)cc2)c1OC(C)=O]

A description of the structure, synthesis, and use of Compound I is found in International Patent Application Number: PCT/US2015/066760, which is hereby incorporated by reference in its entirety along with all the references cited therein. Moreover, Compound I is a known fungicide and provides control of a variety of fungal pathogens in economically important crops including, but not limited to, the causal agent of barley scald, Rhynchosporium secalis (RHYNSE).

One of the preferrable aspects of the present disclosure includes a process to make crystalline Compound I. In some aspects, the process may or may not include transforming amorphous Compound I to crystalline Compound I (in other words, Compound I is crystallized straight from the reaction mixture after the last step of its synthesis, and is not isolated inbetween as a compound. In some aspects, the present disclosure provides a process wherein Compound I is synthesized having an improved impurity profile. In some aspects, the present disclosure provides a process wherein Compound I is more stable and does not degrade or degrades at a much slower rate such that it can be stored and transported.

In some aspects, the present disclosure provides a process wherein crystalline Compound I has the characteristic of being more flowable.

One aspect of the present disclosure includes a method of controlling a pathogen-induced disease in a plant that is at risk of being diseased from the pathogen comprising contacting the plant or an area adjacent to the plant with a composition including one or more crystalline forms of Compound I.

One aspect of the present disclosure includes a use of one or more crystalline forms of compound I for protection of a plant against attack by a phytopathogenic organism or the treatment of a plant infested by a phytopathogenic organism, comprising the application of one or more crystalline forms of compound I, or a composition including one or more crystalline forms of compound I to soil, a plant, a part of a plant, foliage, and/or seeds.

One aspect of the present disclosure includes a composition useful for protecting a plant against attack by a phytopathogenic organism and/or treatment of a plant infested by a phytopathogenic organism comprising one or more crystalline forms of compound I and a phytologically acceptable carrier material.

### Definitions

Various terms used in the specification and claims herein are defined as set forth below, unless otherwise specifically defined in this disclosure. All technical and scientific terms not defined herein have the meaning commonly understood by a person skilled in the art to which this invention belongs.

“Substantially pure or free” refers to a mixture in which one organic compound of interest far exceeds the amount of other small organic compounds in the mixture as impurities, and is at least 80%, 85%, 90%, 95%, at least 96%, at least 97%, at least 98%, at least 99%, at least 99. 5%, at least 99. 7%, or at least 99. 9%, by mol, of the desired compound.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic chemistry.

Throughout this application, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some aspects, ±100%, in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

### DETAILED DESCRIPTION

### Processes

In some aspects, the disclosure provides for a process of making or manufacturing crystalline Compound I. In some aspects, the disclosure provides for a process comprising a. distilling an aprotic organic solvent from a mixture containing Compound I; and b. crystallizing Compound I from a protic organic solvent containing Compound I and seed Compound I, optionally wherein protic solvent is added in step a.

In some aspects, the aprotic organic solvent is dichloromethane. In some aspects, the amount of dichloromethane after step a is: less than 1 wt % and greater than 0.01 wt %, less than 0.5 wt % and greater than 0.005 wt %, less than 1 wt % and greater than 0.005 wt %, less than 0.75 wt % and greater than 0.005 wt %, or less than 1 wt % and greater than 0.001 wt %, residual dichloromethane.

In some aspects, a protic organic solvent is added in step a. In some aspects, the protic organic solvent is isopropyl alcohol.

In some aspects, the water content after step a and before step b is: less than 1000 ppm and greater than 0.1 ppm, 1000 ppm and greater than 50 ppm, less than 400 ppm and greater than 0.1 ppm, less than 400 ppm and greater than 50 ppm, less than 300 ppm and greater than 0.1 ppm, less than 300 ppm and greater than 0.5 ppm, less than 300 ppm and greater than 1 ppm, less than 300 ppm and greater than 10 ppm, less than 300 ppm and greater than 5 ppm, or has a content within a range between any of the numerical concentrations recited herein, i.e. any range between 1000 ppm-400 ppm-300 ppm-50 ppm-10 ppm-5 ppm-1 ppm-0.5 ppm-0.1 ppm, as analyzed by Karl Fisher method.

In some aspects, the amount of Compound I in step a and before step b is: less than 15 wt % and greater than 7 wt %, less than 15 wt % and greater than 10 wt %, less than 10 wt % and greater than 7 wt %, or has an amount within a range between any of the numerical percentages recited herein, e.g., any range between 15 wt %-10 wt %-7 wt %.

In some aspects, the temperature during step a is performed in a range from about 60° C. to about 10° C.

In some aspects, the amount of seed Compound I is: less than or equal to 50 wt % and greater than or equal to 2 wt %, less than or equal to 50 wt % and greater than or equal to 15 wt %, less than or equal to 20 wt % and greater than or equal to 5 wt %, less than or equal to 10 wt % and greater than or equal to 2 wt %, or has an amount within a range of any of the numerical percentages recited herein, e.g., any range from 50 wt %-20 wt %-15 wt %-10 wt %-5 wt %-2 wt %.

In some aspects, the temperature during step b is performed in a range from about 50° C. to about 0° C.

### Impurity Profile and Stability

In some aspects, the disclosure provides for high purity Compound I. In some aspects, the purity is 97.0%, >97.5%, >97.9%, >98.0%, 98.1%, 98.5%, >99.0%, >99.1%, >99.5%, >99.7%, 99.9%, >99.99%, or has a purity within a range of any of the numerical percentages recited herein, i.e. any range from 97.0%-97.5%-97.9%-98.0%-98.1%-98.5%-99.0%-99.1%-99.5%-99.7%-99.9%-99.99%.

In some aspects, the disclosure provides for crystalline Compound I which has increased stability. In some aspects, the crystalline Compound I is stable at 21° C. for greater than 12 hours and less than 24 hours. In some aspects, the crystalline Compound I is stable at 25° C. for greater than 12 hours and less than 24 hours. In some aspects, the crystalline Compound I is stable at 30° C. for greater than 12 hours and less than 24 hours. In some aspects, the crystalline Compound I is stable at 21° C. for greater than 24 hours and less than 36 hours. In some aspects, the crystalline Compound I is stable at 21° C. for greater than 36 hours and less than 48 hours.

### EXAMPLES

The following examples are offered to illustrate this the present technology and are not to be construed in any way as limiting the scope of this the present technology. Efforts have been made to ensure accuracy with respect to numbers used (e.g. amounts, temperatures, etc.), but some experimental error and deviation should, of course, be allowed for. Various modifications of the present technology in addition to those described herein will become apparent to those skilled in the art from the foregoing description and accompanying figures. Such modifications fall within the scope of the appended claims. Unless otherwise stated, all temperatures are in degrees Celsius.

The practice of the present invention will employ, unless otherwise indicated, conventional methods of synthetic organic chemistry, protein chemistry and biochemistry and agriculture are within the skill of the art. Such techniques are explained fully in the literature. See, e.g. T. E. Creighton, Proteins: Structures and Molecular Properties (W. H. Freeman and Company, 1993); A. L. Lehninger, Biochemistry (Worth Publishers, Inc., current addition); Methods In Enzymology (S. Colowick and N. Kaplan eds., Academic Press, Inc.); Remington's Agricultural Sciences, 18th Edition (Easton, Pennsylvania: Mack Publishing Company, 1990); Carey and Sundberg Advanced Organic Chemistry 3^{rd} Ed. (Plenum Press) Vols A and B (1992), and Organic Reactions, Volumes 1-40 (John Wiley, and Sons, 1991).

In the examples below, the following abbreviations have the following meanings. If an abbreviation is not defined, it has its generally accepted meaning.
aq.=aqueous
THF=tetrahydrofuran
o/n=overnight
r. t.=room temperature
DCM=dichloromethane
DMF=dimethylformamide
DMSO=dimethyl sulfoxide
equiv.=equivalent
EtOAc=ethyl acetate
EtOH=ethanol
g=gram
h=hours
HCl=hydrochloric acid
HPLC=high-performance liquid chromatography
M=molar
MeOH=methanol
mg=milligrams
mL=milliliters
mmol=millimols
mp=melting point
wt %=weight percent
μM=micromolar

### General Experimental Details

### General Example: Generic Overview of the Crystallization Process

The reaction mixture (post-work up) typically contains about 2000 ppm of water. Compound I degrades with water present. After the work-up, the organic layer is distilled and then undergoes a solvent exchange. In the present examples reduced to practice, the solvent is dichloromethane (DCM) and the exchange solvent is with isopropyl alcohol (IPA). The typical large-scale, industrial-sized crystallization process—which has been reduced to practice—demonstrates about 75 g-100 kg (93-99%) of Compound I from 60-80 kg crude starting material as an observed as yield out of the crystallization process.

In another generic but more specific example, the process used distillation to first remove the solvent: 80-85% of the feed volume (DCM, water) is distilled overhead under 1 atm. The mixture starts boiling at about 40° C., reaching about 54° C. at the end. This drives off any residual water, typically reducing the water content from around 2000 ppm to <300 ppm water, and of the DCM.

After the DCM distillation, jacket temperature is brought down to 30° C. and room temperature isopropyl alcohol (IPA) (water spec <1000 ppm) is loaded. The vacuum is then set to 180 mm Hg and the jacket temperature to 55° C. Under 180 mm Hg, the mixture starts boiling at around 38° C. and distillation ends at a final bottoms temperature of about 51.3° C. when the bottoms specifications are met (<0.5 wt % residual DCM, 25-27 wt % Compound I).

Following the solvent exchange, the distillation bottoms are cooled to 30° C. and transferred to the crystallizer. If the transfer occurs at a lower temperature, Compound I may come out as oil or amorphous solid. A 5-20% seed loading (seed crystal size D50<20 μm, pretty small, seed loading is pretty high compared to a normal process, which is typically less than 1%) could be used for this process. After seed is loaded, the temperature is held at 30° C. for 2-6 hours and then temperature can be brought down to 8° C. over 4-7 hours.

The crystallization is usually filtered when mother liquor concentration drops below 2.5 wt %. The crystals are filtered and then washed with heptanes. The wet cake is then plowed and scraped into drier where it is dried under a heated vacuum (45 mm Hg and 50° C. max temperature).

Two possible polymorphs are possibly formed from Compound I. Both polymorphic forms are crystalline while amorphous Compound I is not crystalline.

### Example 1: Method of Crystal Formation 1

### Synthesis of Crystalline (S)-1,1-bis(4-fluorophenyl)propan-2-yl (3-acetoxy-4-methoxypicolinoyl)-L-alaninate (Compound I)

### Distillation

Post work-up, the organic layer (723.18 g) containing Compound I was analyzed by Karl Fischer to determine the water content (1620 ppm). A LC sample of the organic layer was taken after the base wash (Compound I wt %=10.89%). The remaining organic layer was then transferred into the distillation vessel. Jacket is set to be 55° C. for the DCM-H₂O distillation. Content temperature rose from 10° C. and started boiling at 40° C. The distillation was stopped at 43° C. (contents temperature) after which point the water content was analyzed by Karl Fisher (257 ppm). The organic layer was cooled to 0° C. and held overnight (16 hours) in the reactor.

The distillation was continued next morning (jacket temperature 65° C.) until the content temperature reached 54° C. Isopropanol (355.05 g, 423 ppm water, 35.1 equiv.) was then added to the bottom. The pressure was set to 180 mmHg (jacket temperature 60° C.). The mixture starts boiling at around 38° C. and distillation ends at a final bottom temperature of about 51.3° C. until the dichloromethane bottom specifications are met (<0.5 wt % residual DCM). The solution was cooled back to 30° C. and was analyzed by HPLC assay—26.4 wt %.

### Crystallization

The post distillation mixture (293.05 g) was then transferred to the crystallizer. Seed slurry (15.04 g solid Compound I polymorph A, purity 97%, active: 14.58 g, d50—10 μm; 45.12 g IPA) was added to the reactor (at 30° C.) and agitated at 500 rpm. The temperature profile used was a gradual step-down over 24 hours from 30° C. to 8° C.

The slurry was filtered when the concentration of Compound I fell below 2.5 wt %. The mother liquor was used to rinse the reactor of solids. The mother liquor was analyzed by HPLC to determine the Compound I content (244.3 g, 2.22 wt % Compound I, active lost 5.42 g). The wet cake was held on the filter for 30 minutes before a heptane wash (102.42 g) was used to rinse the reactor and wash the wet cake. The wet cake (88.42 g) was held on the filter for 30 min and then dried in a vacuum oven at 50° C. overnight to give a colorless solid. Based on XRD the solid formed is polymorph A.

| 2theta | d | Peak height |
|---|---|---|
| 5.97 | 14.792 | 219.1 |
| 10.69 | 8.2692 | 409.8 |
| 14.17 | 6.2452 | 161 |
| 14.71 | 6.0172 | 831.7 |
| 16.19 | 5.4703 | 132.6 |
| 16.99 | 5.2145 | 1000 |
| 17.69 | 5.0097 | 319.7 |
| 18.31 | 4.8414 | 251.8 |
| 19.41 | 4.5695 | 422 |
| 20.01 | 4.4338 | 277.5 |
| 20.67 | 4.2937 | 164 |
| 21.17 | 4.1934 | 229.7 |
| 23.21 | 3.8292 | 803.8 |
| 23.81 | 3.7341 | 313.5 |
| 25.01 | 3.5576 | 185.6 |
| 25.83 | 3.4464 | 134.3 |
| 27.21 | 3.2746 | 138.8 |
| 28.57 | 3.1218 | 168.5 |
| 31.27 | 2.8582 | 94 |
| 32.51 | 2.7519 | 85 |

Wet cake: 88.42 g

Dry cake: 88.38 g

LOD: 0.05 wt % solvent

The dry cake was analyzed by HPLC (97.2 wt %).

active isolated: 85.91 g,

Seed active loaded: 14.58 g

### Example 2: Method of Crystal Formation 2

### Synthesis of Crystalline (S)-1,1-bis(4-fluorophenyl)propan-2-yl (3-acetoxy-4-methoxypicolinoyl)-L-alaninate (Compound I)

### Distillation

Post work-up, the organic layer (727.4 g) was analyzed by Karl Fischer to determine the water content (1852 ppm). A LC sample of the organic layer was taken after the base wash (Compound I wt %=10.95%). The remaining organic layer was then transferred into the distillation vessel.

Jacket is set to be 55° C. for the DCM-H₂O distillation. Content temperature rose from 10° C. and started boiling at 40° C. The distillation was stopped at 43° C. (contents temperature) after which point the water content was analyzed by Karl Fisher (298 ppm). The organic layer was cooled to 0° C. and held overnight (16 hours) in the reactor.

The distillation was continued next morning (jacket temperature 65° C.) until the content temperature reached 54° C. Isopropanol (355.73 g, 355 ppm water) was then added to the bottom. The pressure was set to 180 mmHg (jacket temperature 60° C.). The mixture starts boiling at around 38° C. and distillation ends at a final bottom temperature of about 51.3° C. until the dichloromethane bottoms specifications are met (<0.5 wt % residual DCM). The solution was cooled back to 30° C. and was analyzed by HPLC assay—26.6 wt %.

### Crystallization

The post distillation mixture (298.1 g) was then transferred to the crystallizer. Seed slurry (15.9 g solid Compound I polymorph B, purity 97%, active: 15.42 g, d50—20 μm; 48.2 g IPA) was added to the reactor (at 30° C.) and agitated at 500 rpm. The temperature profile used was a gradual step-down over 24 hours from 30° C. to 8° C.

The slurry was filtered when the concentration of Compound I fell below 2.5 wt %. The mother liquor was used to rinse the reactor of solids. The mother liquor was analyzed by HPLC to determine the Compound I content (215 g, 1.8 wt % Compound I, active lost 3.87 g). The wet cake was held on the filter for 30 minutes before a heptane wash (101.6 g) was used to rinse the reactor and wash the wet cake. The wet cake (88.1 g) was held on the filter for 30 min and then dried in a vacuum oven at 50° C. overnight to give a colorless solid. Based on XRD the solid formed is polymorph B.

| 2theta | d | Peak height |
|---|---|---|
| 7.48 | 11.809 | 68.6 |
| 12.94 | 6.836 | 104.1 |
| 13.52 | 6.544 | 637.8 |
| 14.94 | 5.9251 | 1000 |
| 15.88 | 5.5764 | 200.4 |
| 16.28 | 5.4403 | 165.2 |
| 17.16 | 5.1632 | 222.1 |
| 17.58 | 5.0408 | 84.2 |
| 18.72 | 4.7363 | 582.3 |
| 19.82 | 4.4759 | 518.6 |
| 20.54 | 4.3206 | 581.4 |
| 20.9 | 4.2469 | 38.4 |
| 22.54 | 3.9415 | 461.9 |
| 22.84 | 3.8904 | 464.3 |
| 23.82 | 3.7325 | 274.7 |
| 24.64 | 3.6101 | 299.7 |
| 25.26 | 3.5229 | 127.1 |
| 26.04 | 3.4191 | 166.6 |
| 26.38 | 3.3758 | 309.7 |
| 27.14 | 3.283 | 164.4 |
| 28.46 | 3.1337 | 72.9 |
| 28.74 | 3.1038 | 81.5 |
| 29.46 | 3.0295 | 89.1 |

Wet cake: 88.1 g

Dry cake: 87.2 g

LOD: 0.05 wt % solvent

The dry cake was analyzed by HPLC (97.5 wt %).

active isolated: 85.02 g,

Seed active loaded: 15.42 g

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A process for the preparation of a crystalline compound of the formula: 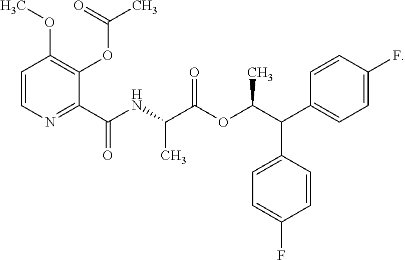 [SMILES: COc1ccnc(C(=O)N[C@@H](C)C(=O)O[C@@H](C)C(c2ccc(F)cc2)c2ccc(F)cc2)c1OC(C)=O]
the process comprising:
distilling a mixture comprising: an aprotic organic solvent, a protic organic solvent, Compound I and seed Compound I.

2. A process for the preparation of a crystalline compound of the formula: 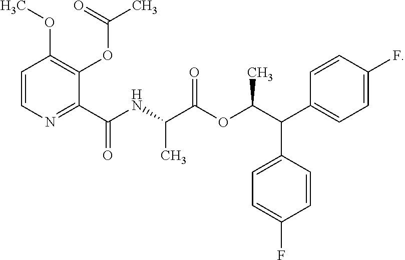 [SMILES: COc1ccnc(C(=O)N[C@@H](C)C(=O)O[C@@H](C)C(c2ccc(F)cc2)c2ccc(F)cc2)c1OC(C)=O]
the process comprising:
a. distilling a mixture comprising: an aprotic organic solvent and Compound I;
b. crystallizing Compound I from step a by creating a second mixture comprising: a protic organic solvent, Compound I and seed Compound I.

3. The process of claim 1 wherein the aprotic organic solvent is dichloromethane.

4. The process of claim 2 wherein the amount of dichloromethane in the mixture after step a is less than 1 wt % and greater than 0.01 wt % residual dichloromethane.

5. The process of claim 2 wherein the amount of dichloromethane in the mixture after step a is less than 0.5 wt % and greater than 0.005 wt % residual dichloromethane.

6. The process of claim 2 wherein a protic organic solvent is added to the mixture in step a.

7. The process of claim 2 wherein the mixture after step a and before step b has a water content of less than 1000 ppm and greater than 50 ppm as analyzed by Karl Fisher method.

8. The process of claim 2 wherein the mixture after step a and before step b has a water content of less than 300 ppm and greater than 0.1 ppm as analyzed by Karl Fisher method.

9. The process of claim 2 wherein the amount of Compound I in the mixture after step a and before step b is less than 15 wt % and greater than 7 wt %.

10. The process of claim 2 wherein the amount of Compound I in the mixture after step a and before step b is less than 15 wt % and greater than 10 wt %.

11. The process of claim 2 wherein the amount of Compound I in the mixture after step a and before step b is less than 10 wt % and greater than 7 wt %.

12. The process of claim 2 wherein the temperature during step a is performed in a range from about 60° C. to about 10° C.

13. The process of claim 2 wherein the amount of seed Compound I is less than 50 wt % and greater than 15 wt %.

14. The process of claim 2 wherein the amount of seed Compound I is less than 10 wt % and greater than 2 wt %.

15. The process of claim 2 wherein the temperature during step b is performed in a range from about 50° C. to about 0° C.

16. A crystalline polymorph of a compound of the formula:

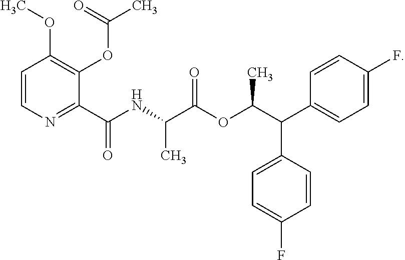 [SMILES: COc1ccnc(C(=O)N[C@@H](C)C(=O)O[C@@H](C)C(c2ccc(F)cc2)c2ccc(F)cc2)c1OC(C)=O]

17. The polymorph of claim 16 wherein the polymorph is characterized by a powder X-ray diffraction pattern having at least the 2θ reflection positions:
2θ
5.97
10.69
14.71
16.99
19.41
23.21.

18. The polymorph of claim 16 wherein the polymorph is characterized by a powder X-ray diffraction pattern having at least the 2θ reflection positions:
2θ
13.52
14.94
18.72
19.82
20.54
22.54
22.84.
